(19) United States
(12) Patent Application Publication
(10) Pub. No.: US 2011/0105984 A1
(43) Pub. Date: May 5, 2011

(54) DUAL LUMEN DIALYSIS CATHETER WITH INTERNALLY BORED OR EXTERNALLY-GROOVED SMALL BORE

(76) Inventors: Aalpen A. Patel, Mt. Laurel, NJ (US); Charles T. Lau, Philadelphia, PA (US)

(21) Appl. No.: 12/936,393

(22) PCT Filed: Apr. 2, 2009
(86) PCT No.: PCT/US09/02047

§ 371 Date: Dec. 28, 2010

Related U.S. Application Data
(60) Provisional application No. 61/064,911, filed on Apr. 2, 2008.
(60) Provisional application No. 61/071,376, filed on Apr. 24, 2008.

(51) Int. Cl. A61M 25/14; A61M 25/04

(52) U.S. Cl. 604/6.16; 604/523; 604/175

(57) ABSTRACT

A dual lumen catheter for insertion into a vein of a patient for use in hemodialysis treatments. An asymmetric dialysis catheter may have a tube containing at least an axial septum asymmetrically disposed within the tube, thereby defining a first and a second lumens, wherein said first lumen is larger in cross sectional area than said second lumen, and a single lumen catheter inserted through the first lumen.

### CROSS REFERENCE TO RELATED APPLICATIONS

This application claims priority to U.S. Provisional Patent Applications 61/064,911, filed Apr. 2, 2008, and 61/071,376, filed Apr. 24, 2008, both of which are incorporated herein by reference in their entirety

### FIELD OF THE INVENTION

The present invention relates to a dual lumen catheter and more particularly to such a catheter for insertion into a vein of a patient for use in hemodialysis treatments.

### BACKGROUND OF THE INVENTION

Hemodialysis is the temporary removal of blood from a patient for the purpose of extracting or separating toxins therefrom and the return of the cleaned blood to the same patient. Haemodialysis is indicated in patients where renal impairment or failure exists, i.e., where the blood is not being properly or sufficiently cleansed, (particularly to remove water) by the kidneys. Dual lumen catheters have been available for many years for a variety of medical purposes and for a number of years such catheters have been used in hemodialysis. The advantage of dual lumen catheters in hemodialysis is that only one vein access need be affected to establish continued dialysis of the blood, as one lumen serves as the conduit for blood flowing from the patient to the dialysis unit and the other lumen serves as a conduit for treated blood returning from the dialysis unit to the patient.

The Kidney Disease Outcomes Quality Initiative (K/DOQI) currently recommends placement of a dual lumen tunneled dialysis catheter in bacteremia-free individuals requiring temporary hemodialysis access. Alternative indications for tunneled dialysis catheter placement include patients who have exhausted all other venous access options and patients requiring hemodialysis while awaiting maturation of their primary fistula. Tunneled dialysis catheters are preferably placed via the right internal jugular vein, with the catheter tip positioned at the caval-atrial junction or within the right atrium in order to optimize flows to and from the venous bloodstream.

Patients requiring tunneled dialysis catheters frequently possess comorbidities requiring extended-duration central venous access. In patients without renal disease, extended-duration central venous access is frequently achieved by the placement of a peripherally inserted central catheter (PICC) via a vein in the upper arm. However, according to the KIDOQI venous preservation doctrine for future arteriovenous access, venipuncture proximal to the dorsum of the hand is relatively contraindicated in the setting of renal insufficiency and/or serum creatinine>3.0 mg/dL. Instead of a PICC, current evidence supports the placement of a tunneled small bore central catheter via an internal jugular vein. In order to lower the risk of tunneled dialysis catheter line infection, such central venous access should be placed independently.

Patients requiring temporary hemodialysis access and extended-duration venous access are currently subject to two independent procedures: placement of a tunneled dialysis catheter and placement of a small bore central catheter. These separate procedures incur additional medical costs and often additional risk. A triple lumen tunneled dialysis catheter could be a solution to this problem. However, current evidence suggests that the additional lumen would increase line infection risk, necessitating removal of the entire catheter. Additionally, it is uncertain if medical personnel managing the third tunneled dialysis catheter lumen would exercise infection control measures equivalent to those of the hemodialysis personnel utilizing the other two lumens.

It is, therefore, desirable to provide a catheter or method for obtaining dialysis of a subject requiring extended duration central venous access that permits: (1) single procedure insertion and (2) an optional extended-duration central venous lumen that may be removed at any time, while leaving the remaining hemodialysis lumens undisturbed.

### SUMMARY OF THE INVENTION

Accordingly, in one embodiment, the invention provides an optional extended-duration central venous lumen that may be removed at any time, while leaving the remaining hemodialysis lumens undisturbed.

In another embodiment, the invention provides an asymmetric dialysis catheter having a tube containing at least an axial septum asymmetrically disposed within the tube, thereby defining a first and a second lumens, and a single lumen catheter inserted through the first lumen. In some embodiments, the first lumen is larger in cross sectional area than the second lumen,

In another embodiment, the invention provides a dialysis catheter having a single tube containing an axial septum disposed therein, thereby defining a first and a second lumens, and a groove extending along the exterior of the tube.

In another embodiment, the invention provides a method for dialysis, in a subject requiring extended-duration central venous access. The method includes the steps of providing a catheter having a tube that contains a first and second lumens; withdrawing blood from a vein of the subject through an inlet aperture of the first lumen of the catheter; and discharging blood from the catheter into the vein through the second lumen of the catheter. In some embodiments, the method further includes the step of inserting a single lumen catheter through the first lumen. In other embodiments, the method further includes the step of inserting a single lumen catheter through a groove, extending along the tube.

The present invention will be better understood by reference to the following detailed discussion of specific embodiments and the attached figures, which illustrate and exemplify such embodiments.

### BRIEF DESCRIPTION OF THE DRAWINGS

Embodiments of the invention will be understood and appreciated more fully from the following detailed description in conjunction with the figures, which are not to scale, in which like reference numerals indicate corresponding, analogous or similar elements.

FIG. 1 shows a cross-sectional view of a first embodiment of the asymmetric tunneled dialysis catheter, according to an embodiment of the invention.

FIG. 2 shows a cross-sectional view of the asymmetric tunneled dialysis catheter with a 4 French small bore central catheter inserted therethrough, according to an embodiment of the invention.

FIG. 3 shows a longitudinal view of a first embodiment of the asymmetric tunneled dialysis catheter, according to an embodiment of the invention.

FIG. 4 shows a longitudinal view of a first embodiment of the single lumen 4 French small bore central catheter, according to an embodiment of the invention.

FIG. 5 shows a longitudinal view of sideport cap for use with the single lumen 4 French small bore central catheter, according to an embodiment of the invention.

FIG. 6 shows a longitudinal view of a first embodiment of the asymmetric tunneled dialysis catheter with single lumen 4 French small bore central catheter installed therein, according to an embodiment of the invention.

FIG. 7 shows a longitudinal view of the asymmetric tunneled dialysis catheter with single lumen 4 French small bore central catheter removed and sideport cap installed, according to an embodiment of the invention.

FIG. 8 shows an enlarged view of the asymmetric tunneled dialysis catheter with a 4 French small bore central catheter inserted therethrough of FIG. 2, according to an embodiment of the invention.

FIG. 9 shows a cross-sectional view of the asymmetric tunneled dialysis catheter having an external groove 3 that extends along the exterior of the tunneled dialysis catheter, according to an embodiment of the invention.

FIG. 10 shows a cross-sectional view of the asymmetric tunneled dialysis catheter with 4 French small bore central catheter 4 inserted into the external groove along the exterior of the tunneled dialysis catheter, according to an embodiment of the invention.

FIG. 11 shows a longitudinal view of the externally-grooved tunneled dialysis catheter, according to an embodiment of the invention.

FIG. 12 shows a longitudinal view of the single lumen 4 French small bore central catheter, according to an embodiment of the invention.

FIG. 13 shows a longitudinal view of the asymmetric tunneled dialysis catheter with single lumen 4 French small bore central catheter installed into the external groove of the tunneled dialysis catheter 17, according to an embodiment of the invention.

FIG. 14 shows the asymmetric tunneled dialysis catheter with a 4 French small bore central catheter inserted into the external groove of the tunneled dialysis catheter of FIG. 9, according to an embodiment of the invention.

FIG. 15 illustrates a method for dialysis, according to an embodiment of the invention.

FIG. 16 illustrates a method for dialysis, according to an embodiment of the invention.

### DESCRIPTION OF THE PREFERRED EMBODIMENT

The following preferred embodiments as exemplified by the drawings are illustrative of embodiments of the invention and are not intended to limit the invention as encompassed by the claims of this application.

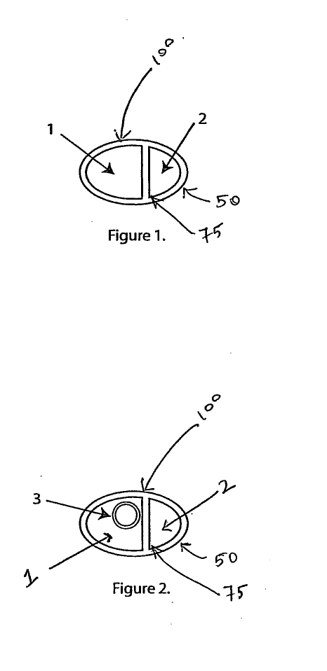

In one embodiment, as shown in FIG. 1, asymmetric tunneled dialysis catheter 50 includes tube 100. Tube 100 may be for example oval or circular in shape. Other suitable shapes may also be used. In one embodiment, tube 100 is a single tube.

Asymmetric tunneled dialysis catheter 50 may have axial septum 75 and two separate lumens 1, 2. In one embodiment, the two lumens are of different cross-sectional area. In certain embodiments, the larger lumen 1 has a cross-sectional area that is greater than the cross-sectional area of the lumen of a typical tunneled dialysis catheter, and the smaller lumen 2 has a cross-sectional area that is equivalent to the cross-sectional area of the lumen of a typical tunneled dialysis catheter. In some embodiments, tube 100 has lumens 1, 2. As shown in FIG. 1, axial septum 75 may be asymmetrically disposed in tube 100.

In another embodiment, lumens 1 and 2 may be of any suitable shape, for example, but not limited to, the shape of a divided circle, a divided oval, a divided ellipse, a divided square, a divided rectangle, a divided bean shape, and a divided crescent shape. In a particular embodiment, lumens 1 and 2 are in D-shape. In some embodiments, Lumens 1 and 2 are in same or similar shape. In other embodiments, Lumens 1 and 2 are in different shapes.

Asymmetric tunneled dialysis catheter 50 may include a single lumen catheter inserted through at least one of lumens 1, 2. The suitable single lumen catheter may include, for example, a single lumen 4 French small bore central catheter or a single lumen 5 French small bore central catheter. Other suitable single lumen catheters may also be used.

As shown in FIG. 2, a single lumen 4 French small bore central catheter 3 may be inserted through asymmetric tunneled dialysis catheter 50, preferably through the larger lumen 1 thereof. In one embodiment, with the small bore central catheter 3 inserted, the remaining cross-sectional area of the larger lumen 1, i.e., the cross-sectional area of the larger lumen 1 less the cross-sectional area of the small bore central catheter 3, is substantially equivalent to the cross-sectional area of the smaller lumen 2. Other catheters or lumens, having sizes other than a 4 French small bore central catheter, may be used.

In one embodiment, the term “4 French small bore central catheter ” refers to a 4 F catheter, or a 5 F catheter in another embodiment, where the catheters described herein encompass both single or double lumen catheters.

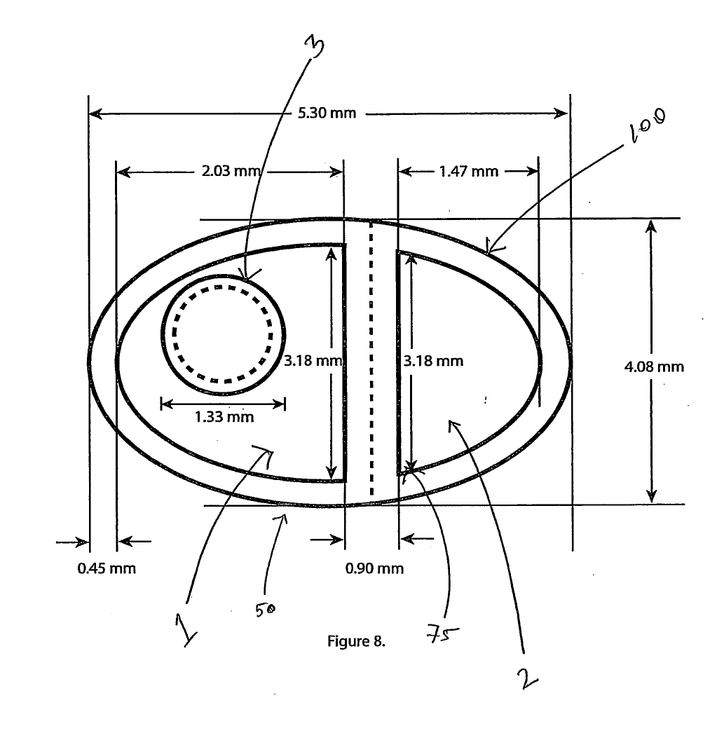

FIG. 8 shows cross-sectional dimensions of one embodiment of asymmetric tunneled dialysis catheter 50 with 4 French small bore central catheter inserted. In this embodiment, the catheter is of elliptical shape, with external width of approximately 5.30 mm and external height of approximately 4.08 mm. In this embodiment, the catheter wall thickness is approximately 0.45 mm, and the septum between the two lumens 1,2 is approximately 0.90 mm, and the internal height is approximately 3.18 mm. In this embodiment, the diameter of the small bore central catheter is approximately 1.33 mm. Other dimensions and ratios may be used.

In some embodiments, asymmetric tunneled dialysis catheter 50 has uniform outside diameter. In one embodiment, a ratio of an outer wall thickness of the catheter to the outside diameter is from about 0.01 to about 0.2. In another embodiment, a ratio of thickness of a septum to the outside diameter is from about 0.02 to about 0.4. In another embodiment, the aspect ratio between the long and short diameters of a oval tube 100 is between about 1.0 and about 1.5.

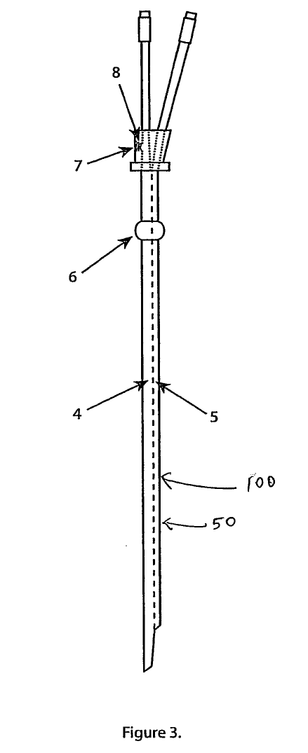

In one embodiment, as shown in FIG. 3, asymmetric tunneled dialysis catheter 50 has two lumens of different cross-sectional area: a larger lumen 4 and a smaller lumen 5. In certain embodiments, a retention cuff 6 is integrated along the shaft of the catheter. In certain embodiments, a threaded sideport 7 communicates with the larger lumen of asymmetric tunneled dialysis catheter 50. In a preferred embodiment, this sideport 7 is wholly contained within the reinforced junctional portion of the dialysis catheter. In one embodiment, as a protective measure, the threading 8 of this sideport 7 will be incompatible with the Luer-type threading universally used in clinical settings. This incompatibility may discourage violation of this sideport 7 after installation of the dialysis catheter system. In one embodiment there is no communication between the threaded sideport 7 and the smaller lumen 2 of asymmetric tunneled dialysis catheter 50.

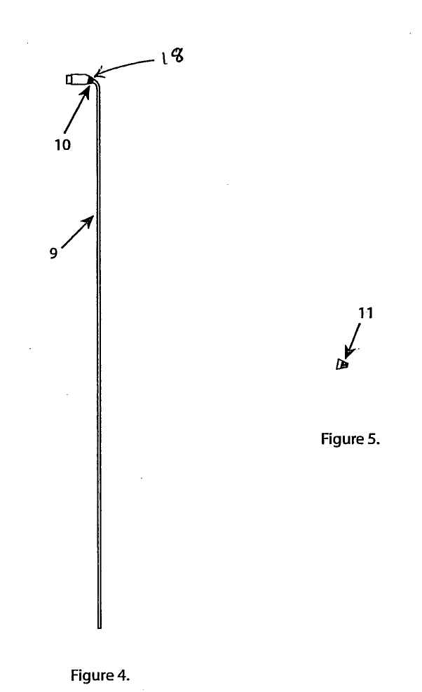

FIG. 4 shows a longitudinal view of a single lumen 4 French small bore central catheter. The 4 French small bore central catheter has a single lumen 9 and a threaded hub 10 that is designed to mate with the threaded sideport 7, preferably in a threaded relationship, communicating with the larger lumen 1 of asymmetric tunneled dialysis catheter 50. In certain embodiments, the threading 18 on this hub 10 is compatible with the threading 8 of the sideport 7, but incompatible with the Luer-type threading universally used in clinical settings.

An embodiment of an occlusive sideport cap 11 that is designed to mate with the threaded sideport 7 communicating with the larger lumen of asymmetric tunneled dialysis catheter 50, is shown in FIG. 5. The occlusive sideport cap preferably has a threaded hub that is designed to screw into the threaded sideport 7. In certain embodiments, the threading on this hub is compatible with the threading of the sideport 7, but incompatible with the Luer-type threading universally used in clinical settings.

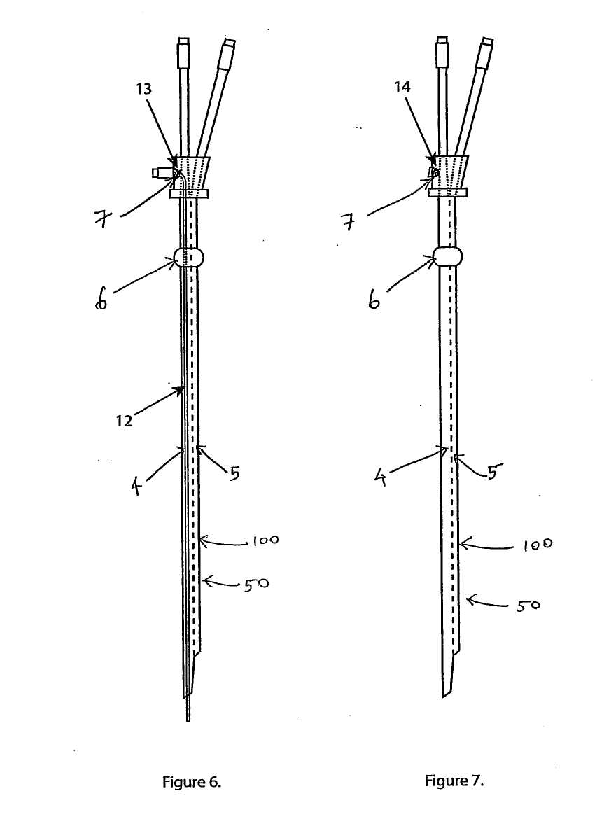

Asymmetric tunneled dialysis catheter 50 with single lumen 4 French small bore central catheter installed therein is shown in FIG. 6. In certain embodiments, the single lumen 4 French small bore central catheter is introduced via the threaded sideport 7 of the asymmetric tunneled dialysis catheter, through the larger lumen 12, and out of the tip of the dialysis catheter. It is secured once its hub 10 has been screwed into the threaded sideport 13. FIG. 7 shows asymmetric tunneled dialysis catheter 50 with single lumen 4 French small bore central catheter removed and sideport cap installed. The threaded sideport 7 is preferably occluded using the sideport cap 14.

One advantage of this structure may be that the single lumen 4 French small bore central catheter may be removed at any time without requiring removal of asymmetric tunneled dialysis catheter 50.

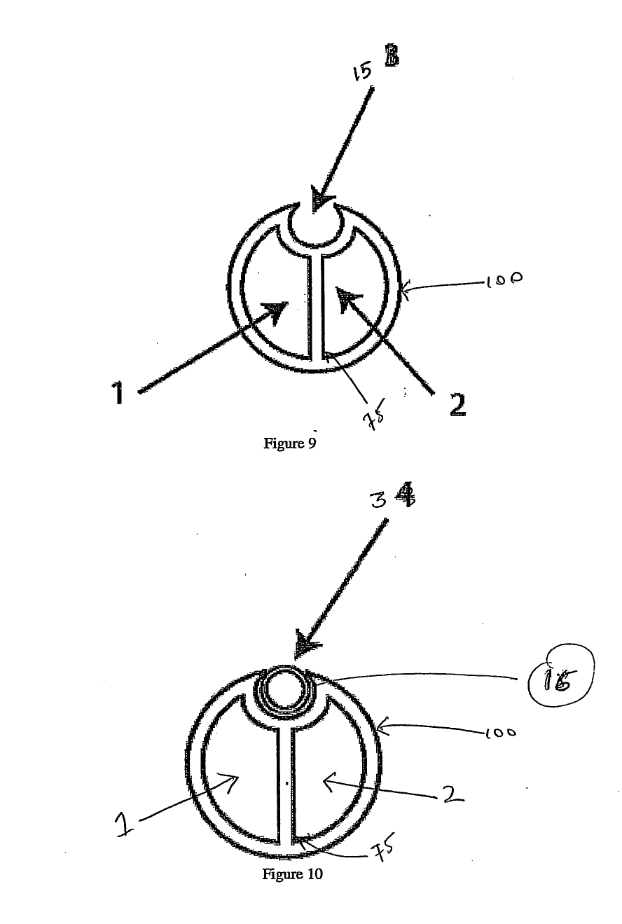

In one embodiment of the invention, as shown in FIG. 9, an externally-grooved tunneled dialysis catheter has two separate lumens 1, 2. In one embodiment, the two lumens are of different cross-sectional area, as discussed herein. In certain embodiments, the larger lumen 1 has a cross-sectional area that is greater than the cross-sectional area of the smaller lumen 2. In another embodiment, the two lumens are of substantially equal cross-sectional area. In a further embodiment, each lumen has a cross-sectional area equivalent to a lumen of a typical tunneled dialysis catheter.

In this embodiment, the single lumen 4 French small bore central catheter 3 is not inserted through the tunneled dialysis catheter. Instead, the tunneled dialysis catheter has an external groove 15 extends along the exterior surface thereof. FIG. 10 shows the externally-grooved tunneled dialysis catheter with a 4 French small bore central catheter inserted into the external groove along the exterior of the tunneled dialysis catheter. In certain embodiments, small lips 16 are present along the edges of this groove, in order to aid in the retention of the small bore central catheter. The cross-section of external groove 15 may be of any suitable shape, for example, but not limited to, circle, oval, ellipse, square rectangle, a bean shape, and a crescent shape.

FIG. 14 shows cross-sectional dimensions of one embodiment of the tunneled dialysis catheter with 4 French small bore central catheter inserted. In this embodiment, the catheter is of circular shape, with external diameter of approximately 4.96 mm. In this embodiment, the catheter wall thickness is approximately 0.45 mm, and the septum between the two lumens 1, 2 is approximately 0.90 mm. In this embodiment, the diameter of the external groove is approximately 1.33 mm. Other suitable dimensions may be used.

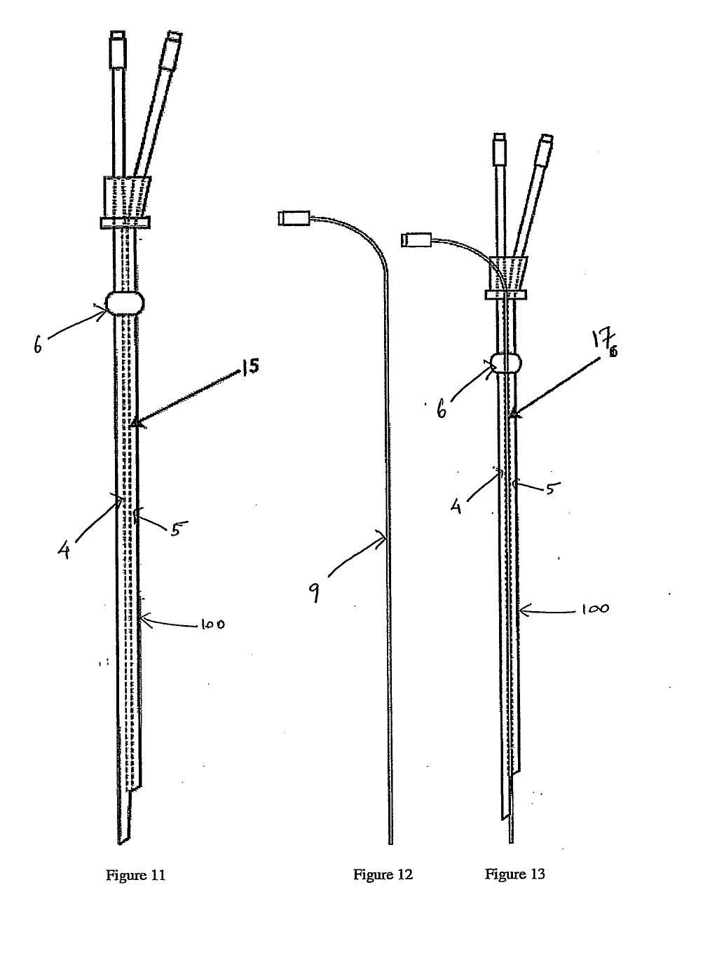

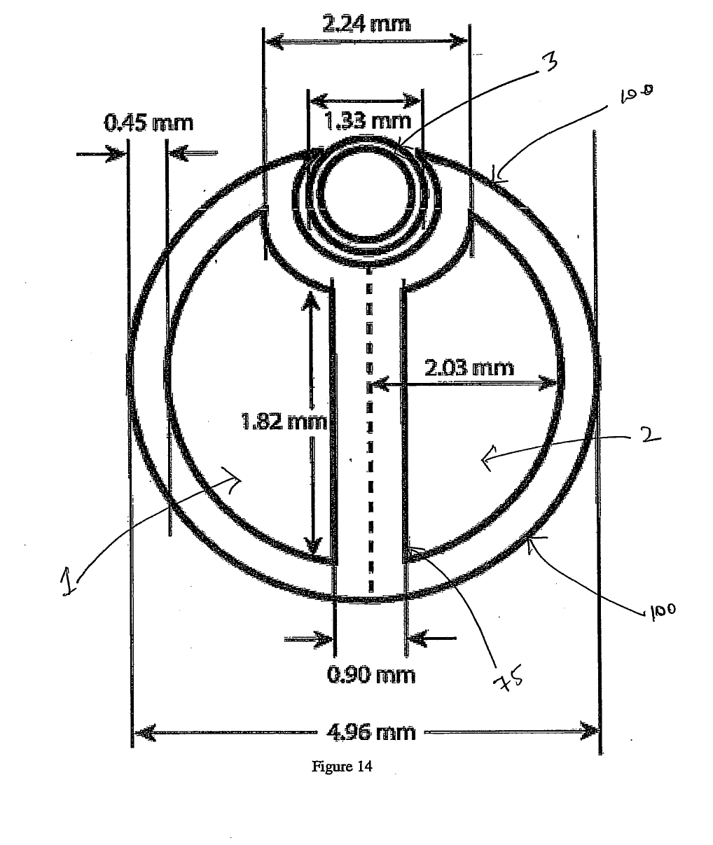

FIG. 11 shows a longitudinal view of the externally-grooved tunneled dialysis catheter, with an external groove 5 extending along the exterior of the tunneled dialysis catheter. The single lumen 4 French small bore central catheter, shown in FIG. 12, has a single lumen and is of sufficient diameter to fit securely into the external groove of the tunneled dialysis catheter. FIG. 13 shows asymmetric tunneled dialysis catheter 50 with single lumen 4 French small bore central catheter installed and secured into the external groove of the tunneled dialysis catheter 6.

In this configuration, the dialysis catheter system may now be installed within a patient. If necessary, the single lumen 4 French small bore central catheter may be withdrawn at any time in the future, while leaving the tunneled dialysis catheter intact.

In another embodiment, the invention provides a method for dialysis using asymmetric tunneled dialysis catheter 50. In one embodiment, the method is performed on a subject who requires extended-duration central venous access. In another embodiment, the method is performed on a subject who has renal insufficiency. In yet another embodiment, the method is performed on a subject whose creatinine level is no less than 3.0 mg/dL of blood.

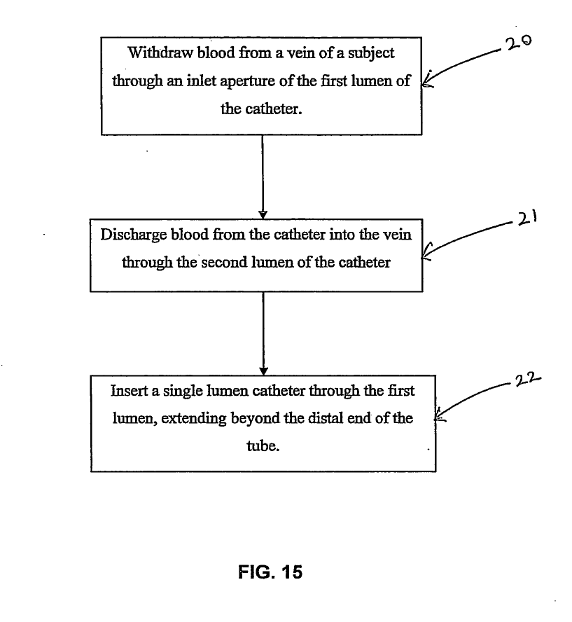

FIG. 15 illustrates a method for dialysis, using asymmetric tunneled dialysis catheter 50 having tube 100 that includes lumens 1 and 2, according to one embodiment of the invention. As shown in FIG. 15, item 20, in one embodiment, blood is withdrawn from a vein of a subject through an inlet aperture of lumen 1 of the catheter. As shown in item 21, blood is discharged from the catheter into the vein through lumen 2 of the catheter. As shown in item 22, a single lumen catheter is inserted through lumen 1, extending beyond the distal end of tube 100. Other operations or series of operations may be used, and the embodiment of FIG. 15 may be used with devices described herein, but need not be.

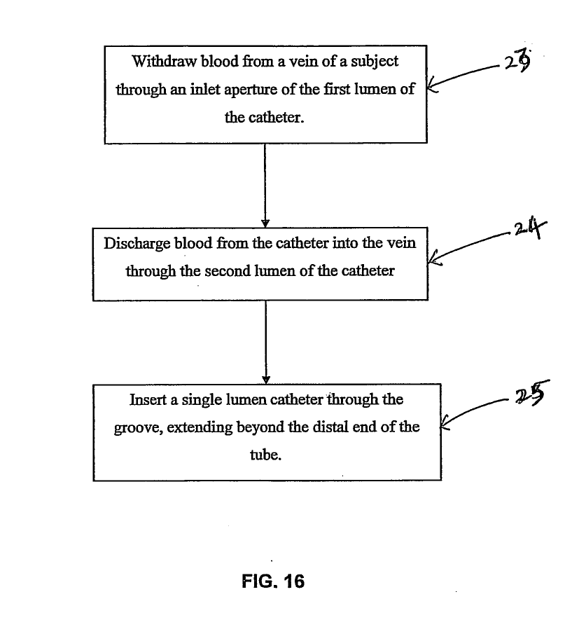

FIG. 16 illustrates a method for dialysis, using asymmetric tunneled dialysis catheter 50 having tube 100 that includes lumens 1 and 2 and groove 15 extending along tube 100, according to another embodiment of the invention. As shown in FIG. 16, item 23, in one embodiment, blood is withdrawn from a vein of a subject through an inlet aperture of lumen 1 of the catheter. As shown in item 24, blood is discharged from the catheter into the vein through lumen 2 of the catheter. As shown in item 25, a single lumen catheter is inserted through groove 15, extending beyond the distal end of tube 100. Other operations or series of operations may be used, and the embodiment of FIG. 16 may be used with devices described herein, but need not be.

In some embodiments, the method for dialysis includes the step of capping sideport 7 communicating with said first lumen. After dialysis, in one embodiment, the single lumen catheter is removed from asymmetric tunneled dialysis catheter 50.

Various embodiments of a catheter have been provided. One skilled in the art will appreciate that the present invention can be practiced by other than the described embodiments, which are presented for purposes of illustration and not limitation, and that the invention is limited only by the claims that follow. Further, since numerous modifications and changes will readily occur to those skilled in the art, it is not desired to limit the invention to the exact construction and operation shown and described, and accordingly, all suitable modifications and equivalents may be resorted to, without departing from the scope or spirit of the invention as defined in the appended claims.

What is claimed is:

1. An asymmetric dialysis catheter having a distal end and a proximal end, the catheter comprising:
a tube containing at least an axial septum asymmetrically disposed within the tube, thereby defining a first and a second lumens, wherein said first lumen is larger in cross sectional area than said second lumen, and
a single lumen catheter inserted through said first lumen.

2. The catheter of claim 1, wherein said tube is a oval tube.

3. The catheter of claim 1, wherein said tube is a single oval tube.

4. The catheter of claim 1, wherein said first lumen is a D-shaped lumen.

5. The catheter of claim 1, wherein said second lumen is a D-shaped lumen.

6. The catheter of claim 1, wherein said single lumen catheter is 4 or 5 French small bore central cathter.

7. The catheter of claim 1, having uniform outside diameter.

8. The catheter of claim 7, wherein a ratio of an outer wall thickness of said catheter to said outside diameter is from about 0.01 to about 0.2.

9. The catheter of claim 7, wherein a ratio of thickness of said septum to said outside diameter is from about 0.02 to about 0.4.

10. The catheter of claim 2, wherein the aspect ratio between the long and short diameters of said oval tube is between about 1.0 and about 1.5

11. The catheter of claim 1, further comprising a retention cuff integrated along the tube of the catheter.

12. The catheter of claim 1, further comprising a threaded sideport communicating with the larger lumen.

13. The catheter of claim 12, wherein the sideport is wholly contained within a reinforced junctional portion of the catheter.

14. The catheter of claim 12, wherein the threading of the sideport is incompatible with Luer-type threading.

15. The catheter of claim 12, further comprising a threaded hub adapted to attach to the threaded sideport.

16. The catheter of claim 15, wherein the threaded hub is incompatible with Luer-type threading.

17. The catheter of claim 1, further comprising an occlusive sideport cap having a threaded hub designed to attach to the threaded sideport,

18. The catheter of claim 17, wherein the threaded hub of the occlusive cap is incompatible with Luer-type threading.

19. The catheter of claim 6, wherein the single lumen 4 French small bore central catheter inserted through the larger lumen extends beyond the distal end of the single tube.

20. The catheter of claim 2, wherein said first and second lumen extend beyond the oval tube, thereby forming a first D-shape lumen and a second D-shape lumen.

21. A dialysis catheter having a distal end and a proximal end comprising:
a single tube containing an axial septum disposed therein, thereby defining a first and a second lumens, and
a groove extending along the exterior of said tube.

22. The catheter of claim 21, wherein the outside lumen has a substantially circular groove defined therein, said groove is adapted to substantially enclose an external device therein, said grove extending axially along said axial septum.

23. A dialysis system, comprising:
a dialysis unit; and
a catheter coupled to said dialysis unit, wherein said catheter comprises a tube that comprises a first and second lumens, and wherein a single lumen catheter inserted through said first lumen.

24. A dialysis system, comprising:
a dialysis unit; and
a catheter coupled to said dialysis unit, wherein said catheter comprises a tube that comprises a first and second lumens and a groove extending along said tube, and wherein a single lumen catheter inserted through said groove.

25. A method for dialysis, in a subject requiring extended-duration central venous access, the method comprising:
providing a catheter, wherein said catheter comprises a tube that comprises a first and second lumens;
withdrawing blood from a vein of said subject through an inlet aperture of said first lumen of said catheter;
discharging blood from said catheter into said vein through said second lumen of said catheter; and
inserting a single lumen catheter through said first lumen, extending beyond the distal end of said tube.

26. The method of claim 25, wherein the subject has renal insufficiency.

27. The method of claim 25, wherein creatinine level of the subject is no less than 3.0 mg/dL of blood.

28. The method of claim 25, further comprising the step of removing the single lumen catheter and capping a sideport communicating with said first lumen.

29. A method for dialysis, in a subject requiring extended-duration central venous access, the method comprising:
providing a catheter, wherein said catheter comprises a tube that comprises a first and second lumens and a groove extending along said tube;
withdrawing blood from a vein of said subject through an inlet aperture of said first lumen of said catheter;
discharging blood from said catheter into said vein through said second lumen of said catheter; and
inserting a single lumen catheter through said groove, extending beyond the distal end of said tube.

30. The method of claim 29, wherein the subject has renal insufficiency.

31. The method of claim 29, wherein creatinine level of the subject is no less than 3.0 mg/dL of blood.

32. The method of claim 29, further comprising the step of removing the single lumen catheter and capping a sideport communicating with said first lumen.

33. The method of claim 29, wherein the first lumen is the smaller lumen.
